(19) United States
(12) Patent Application Publication
(10) Pub. No.: US 2018/0303456 A1
(43) Pub. Date: Oct. 25, 2018

(54) PORTABLE MEDICAL STATION

(71) Applicants: Jack Tawil, BROOKLYN, NY (US); David Jack Tawil, Brooklyn, NY (US); Christopher Smith, Shirley, NY (US); Holland Bennett, Syosset, NY (US)

(72) Inventors: Jack Tawil, BROOKLYN, NY (US); David Jack Tawil, Brooklyn, NY (US); Christopher Smith, Shirley, NY (US); Holland Bennett, Syosset, NY (US)

(21) Appl. No.: 15/953,417

(22) Filed: Apr. 14, 2018

Related U.S. Application Data
(60) Provisional application No. 62/488,048, filed on Apr. 20, 2017.
(60) Provisional application No. 62/488,735, filed on Apr. 22, 2017.

(51) Int. Cl. A61B 8/00; A61B 7/02; A61B 5/01; A61B 5/00; A61B 5/021; A61B 5/0245; A61B 50/33; G06F 1/16

(52) U.S. Cl. CPC A61B 8/4427; A61B 7/02; A61B 5/01; A61B 5/4803; A61B 5/021; A61B 2050/3011; A61B 5/7445; A61B 50/33; G06F 1/1647; G06F 1/1628; A61B 5/0245

(57) ABSTRACT

At least one embodiment is a portable medical station comprising a main body, which is disposed at least partially inside of a bag. Coupled to the main body is at least one tray and a plurality of handles. There are also a plurality of screens coupled to the main body and at least one computer coupled to the main body, wherein the portable medical station is foldable inside of the bag so that the portable medical station is movable while being substantially folded inside of the bag.

### CROSS REFERENCE TO RELATED APPLICATIONS

This application is a non-provisional application that hereby claims priority from U.S. Provisional Application Ser. No. 62/488,735 filed on Apr. 22, 2017, and also from U.S. Provisional Application Ser. No. 62/488,048 filed on Apr. 20, 2017, the disclosures of which are hereby incorporated herein by reference in their entirety.

### BACKGROUND OF THE INVENTION

At least one embodiment of the invention relates to a portable medical station configured to be collapsible into a single unit or which is capable of folding out into a more expansive station. It is believed that there is a need for a portable medical station that is configured to have multiple different screens and multiple different portable devices coupled to the portable medical station.

### SUMMARY OF THE INVENTION

At least one embodiment of the invention is a portable medical station comprising a main body, which is disposed at least partially inside of a bag. Coupled to the main body is at least one tray and a plurality of handles. There are also a plurality of screens coupled to the main body and at least one computer coupled to the main body, wherein the portable medical station is foldable inside of the bag so that the portable medical station is movable while being substantially folded inside of the bag.

In at least one embodiment, the tray is rotatable about a hinge wherein the tray is foldable out from, or back into the main body. In at least one embodiment, there are a plurality of screens which comprise a first screen which is configured as a video screen coupled to the computer, and a second screen comprising a data readout screen coupled to the computer.

In at least one embodiment there is a spring configured to assist in the opening of the medical station from a bag.

In at least one embodiment, the main body comprises a frame which can be made from any material, but in at least one embodiment is aluminum.

In at least one embodiment, there is at least one peripheral device wherein the peripheral device can be any one of a stethoscope a thermometer, an ultrasound device, at least one camera, at least one speaker, and/or at least one blood pressure monitor.

In at least one embodiment, there is a plurality of arms extending out from the main body wherein a first end of the arms are coupled to the main body and a second end of the plurality of arms are coupled to the tray.

For example, the process can include opening a bag, and then removing at least one scale from the bag. Next the process can include pulling a handle and unfolding a tray from the bag. When the handle is pulled a spring such as a gas spring can be used to assist in the opening of the device from the bag. The tray can be rotated about a hinge and then locked in place. Supporting the tray are a plurality of arms extending up from the main body of the device. These plurality of arms can be locked in place via locks. Next, the user can raise at least one screen up and out from the bag. The screen can be rotated about a hinge and then locked in place by rotating a plurality arms out to a locked position. Next a main body can be raised up thereby raising a plurality of peripheral devices up and out from the bag. At least one camera can be placed upon a viewing position such as on top of a screen so that a remote medical professional can view a patient in a remote location. There can also be a process for collapsing the different devices into the bag as well. For example, the handles can be collapsed into the bag by pressing on one or more spring loaded detents. Next, the tray can be collapsed into the main body by pressing on at least one lever, which causes the plurality of locks to concurrently unlock the arms. This causes the tray to collapse into the main body. Next, the screen can be rotated down and collapsed into the main body and the main body can be pressed into the remainder of the bag. Finally, the scale can be reinserted into at least one pocket on the bag as well to create a completely collapsed medical station.

Thus, this compact, portable device is configured to be connected to or connectable to multiple different peripheral devices while also being highly transportable as well. This device then allows medical professionals to conduct a relatively thorough exam on an individual. The camera in the system allows for remote medical professionals to interview and see a patient from a remote location as well.

With this design, full communication can be made between the patient and medical professionals who may be remote from this location. Therefore, it allows for a portable medical office to be transported within a standard sized bag such as a standard sized carry on bag.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a front view of the device with a front flap on the cover folded down;

FIG. 2 is a front view of the device in an expanded form;

FIG. 3 is a front-right side perspective view of the device in an expanded form;

FIG. 4 is a front-left side perspective view of the device in an expanded form;

FIG. 5 is a front-left side perspective view of the device in an expanded form;

FIG. 6 is a side view of the device in an expanded form;

FIG. 7 is a side view of the device in a collapsed form;

FIG. 8 is a schematic block diagram of some of the electrical components coupled to the device;

FIG. 9 is a front view of the device without the screens;

FIG. 10 is a side view of the device without the screens

FIG. 11 is a front-right side view of the device without the screens;

FIG. 12 is a front collapsed view of the device with the screens;

FIG. 13 is a side collapsed view of the device;

FIG. 14 is a top front perspective view of the device;

FIG. 15 is a side view of another embodiment of a portable medical device;

FIG. 16 is a front view of the embodiment of FIG. 15;

FIG. 17 is a perspective view of the embodiment of FIG. 15; and

FIG. 18 is a view of the portable medical station positioned inside of a bag.

### DETAILED DESCRIPTION OF PREFERRED EMBODIMENTS

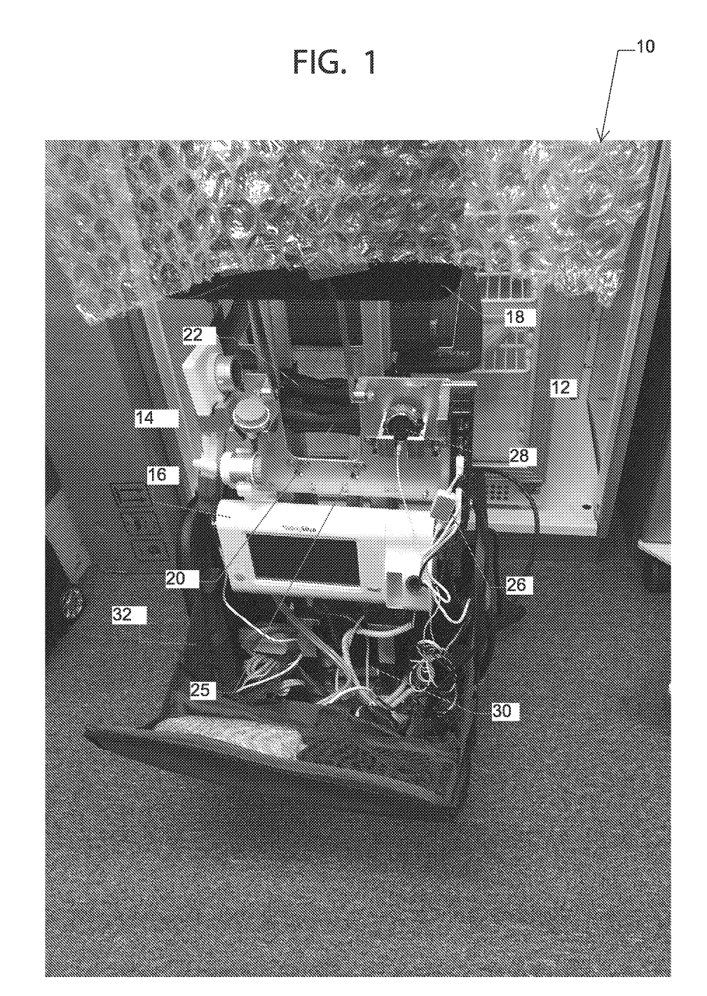

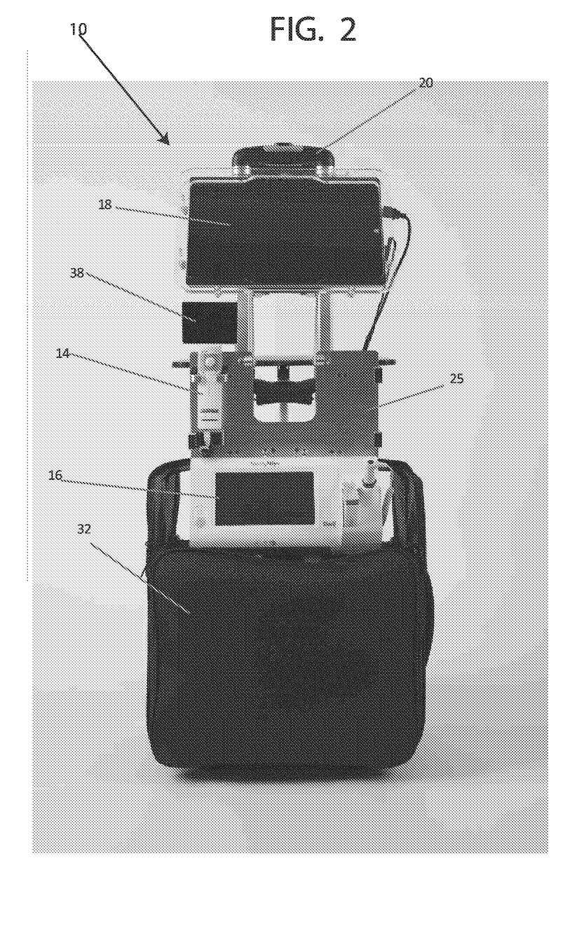

Referring to the drawings, FIG. 1 shows a front view of the device 10 which shows a main section 12 having a plurality of different components. For example, there is shown at least one peripheral device 14 which can be configured as a ultrasound gauge. In addition, there is a first screen 16 which is configured to provide data to a user such as a medical professional. This first screen 16 is configured to have data readouts such as readouts from an EKG shown on this screen. In addition, there is a second or additional screen 18 as well. This screen 18 is configured as a more standard computer and or television screen which can be in the form of a standard LED or LCD screen. In addition, there are also a plurality of handles such as handles 20 and 22. These handles 20 and 22 are shown in a collapsed manner in FIG. 1, but are shown in an expanded form in FIGS. 2-6. Handle 20 is a first back handle and is collapsible in a telescoping manner. Handle 22 is a second handle positioned in an intermediate position between screen 18 and handle 20 and which allows for the control of the device when it is being dragged or rolled around. The second handle together with the first handle provides additional stability for the device and prevent it from toppling over when it is being dragged or pulled. In addition, there is shown a substantially U-shaped frame 25 which can be made from any suitable material but in this example is made of metal. In addition, there is shown an additional peripheral device 26 which comprises an annunciator or speaker for allowing individuals to speak through this speaker.

A further peripheral device 28 is also shown, showing a stethoscope which is also coupled to a main computing element which is stored inside of a housing behind main screen 16. Much of the device resides in a lower section 30 which is disposed normally inside of a bag such as bag 32.

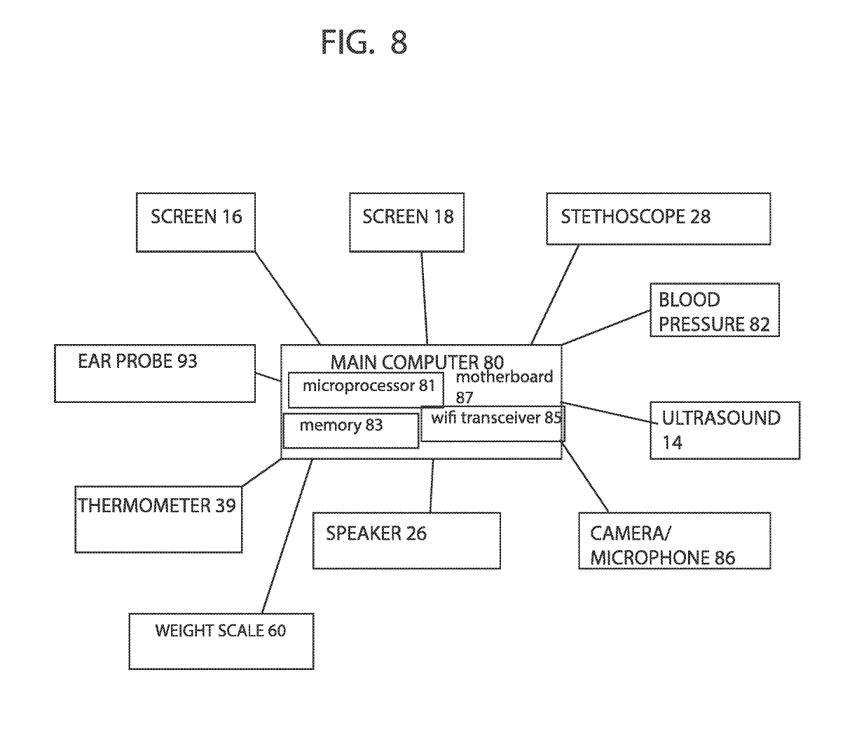

FIG. 2 shows a front view of the device 10 which shows bag 32 with the device being expanded out of the bag. There is a screen 18 which is shown expanded up out of the bag, and a data screen 16 which is also shown expanded up out of the bag as well. Handles 20 and 22 are shown extending above screen 18. An additional screen 38 is shown coupled to peripheral device/ultrasound 14. Screen 16 is shown below these devices as well. All of these components are coupled to a central computer or main computer 80 (See FIG. 8).

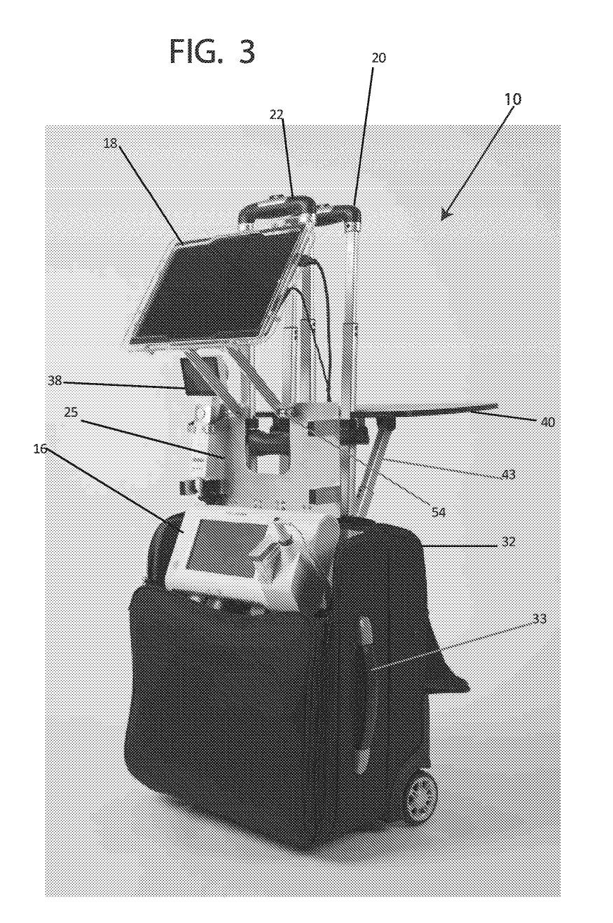

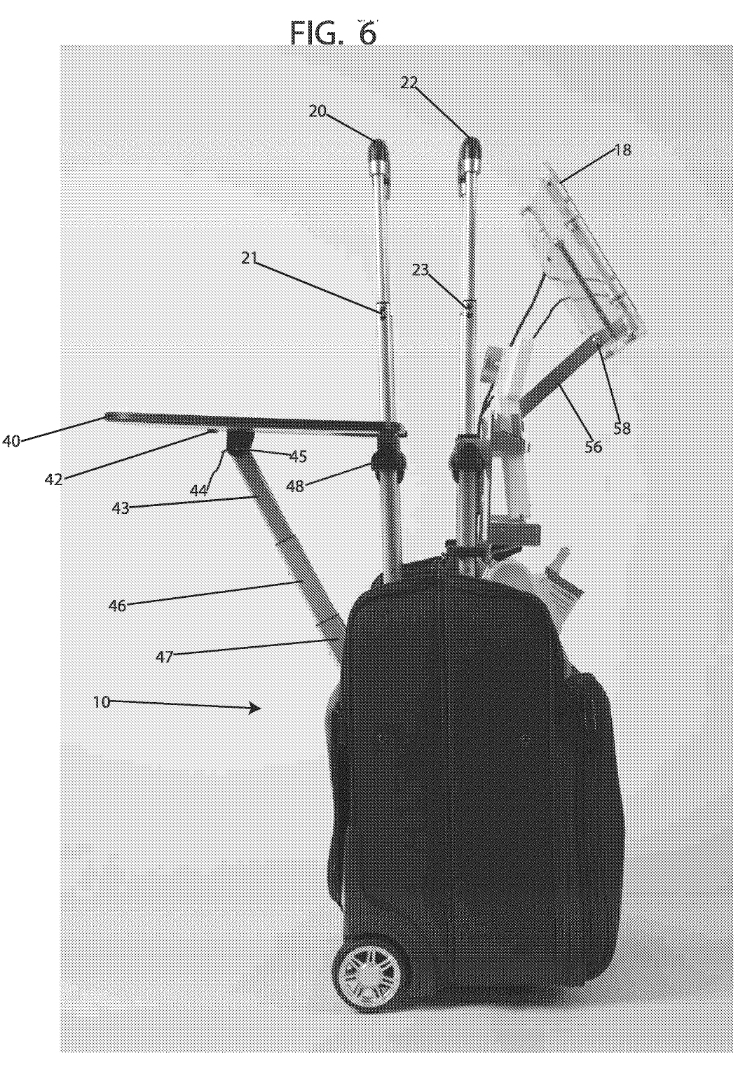

FIG. 3 is a front-side perspective view of the device 10. As shown in this view there are handles 20 and 22 which are shown extended up above the screen 18. An additional screen 38 is shown below screen 18. A back tray 40 is shown extending out from the back face of the device 10. Back tray 40 is supported by arms such as arm 43. The screen 18 is supported by arms which are rotatable about a hinge 54. The arm supporting screen 18 is arm 56 shown in greater detail in FIG. 6 and extends up to hinge 58. Hinge 58 is coupled to screen 18 as well. As shown in this view, a frame 25 is shown positioned between screen 18 and screen 16. This device is positioned inside of a bag 32 having a handle 33.

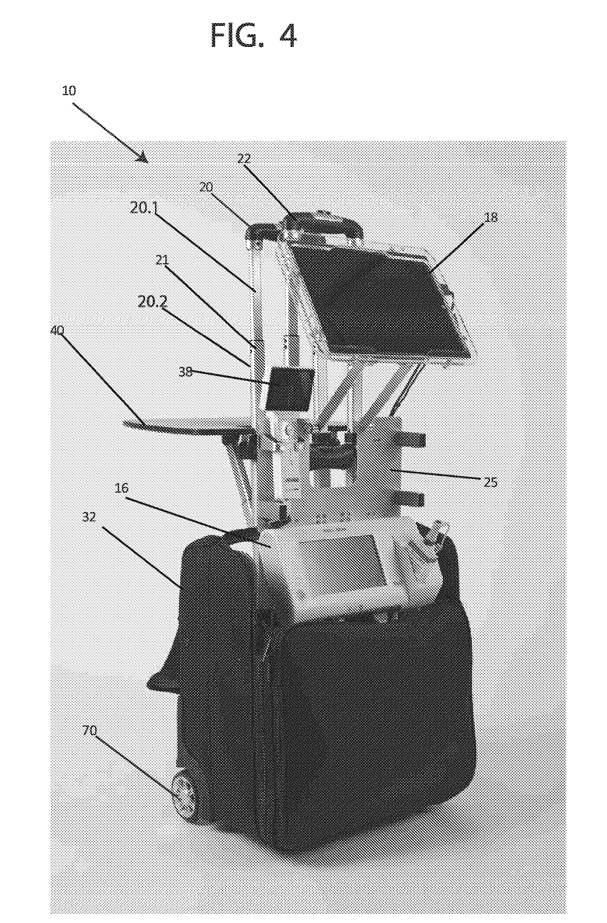

FIG. 4 is a front left side perspective view of the device 10 showing handles 20 and 22. These handles 20 and 22 are telescoping and collapsible. For example, handle 20 includes a first section 20.1 a lock 21, which includes spring loaded detents for selectively locking the collapse of these arms such as the collapse of first section 20.1 into second section 20.2 These arms extend up opposite screen 18, and above tray 40.

Tray 40 extends out opposite the extension of screen 18. Screen 16 sits below screen 18 and sits opposite handles 20 and 22. Bag 32 is shown enclosing most of the lower section of the device 10 and this device 10 includes wheels 70 as well.

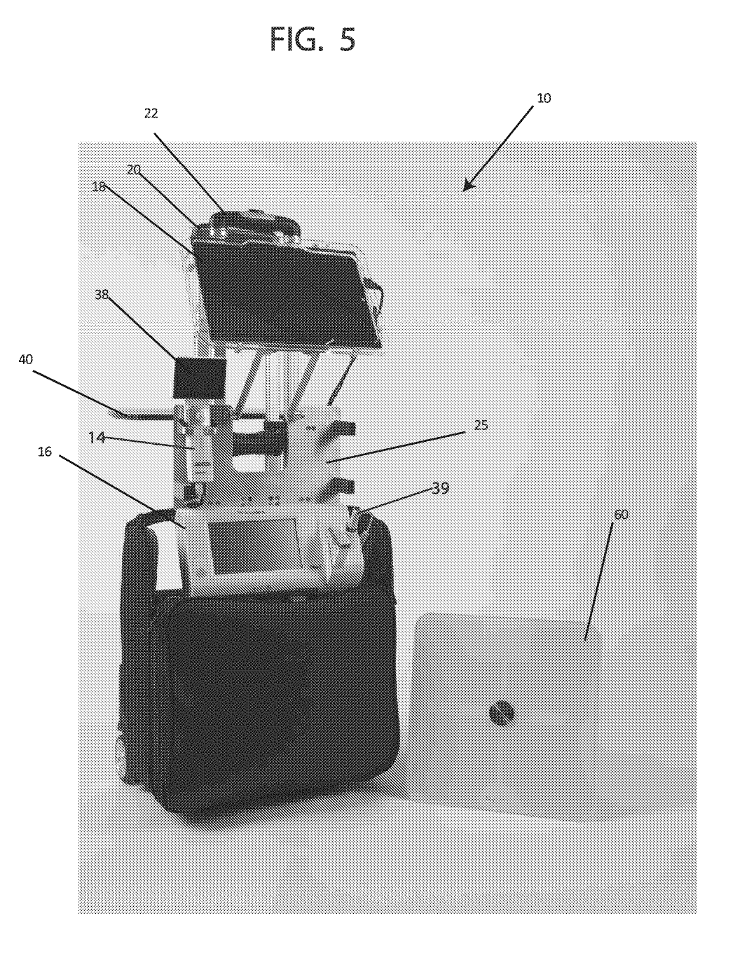

FIG. 5 is a front left view of the device with the portable weight scale 80 shown as well. Handles 20 and 22 are shown, as well as screens 16, 18 and 38. Screen 38 is configured to show the results of the ultrasound device 14. Tray 40 is shown extending out from the back of the device. Frame 25 is shown connecting upper screen 18 to lower screen 16.

FIG. 6 shows a side view of the device 10. In this view, there is shown handles 20 and 22 which each have selective locks 21 and 23 for selectively allowing these handles 20 and 22 to collapse. Each of these locks 21 and 23 are configured as spring loaded detents that when pinched in allow the handles to collapse. Screen 18 is shown extending up and connected to the main body via arm 56 and hinge 58. Arm 56 is coupled to frame 25. Back tray 40 is coupled to the main body via arms 43, 46, and 47 which are selectively collapsible via locks such as spring loaded detents as well. In a region near the end of arm 43 is a hinge 45 which is coupled to tray 40. A lever 42 is shown positioned below tray 40 which when pressed, sets the locks to an open position for arms 43, 46, and 47, allowing tray 40 to collapse into the bag. A tab 44 is also extending out from hinge 45. This hinge 45 is configured to unlock the locks when tripped by the lever 42 as well. This unlocking feature allows for the rapid collapse of tray 40 into the body of the device.

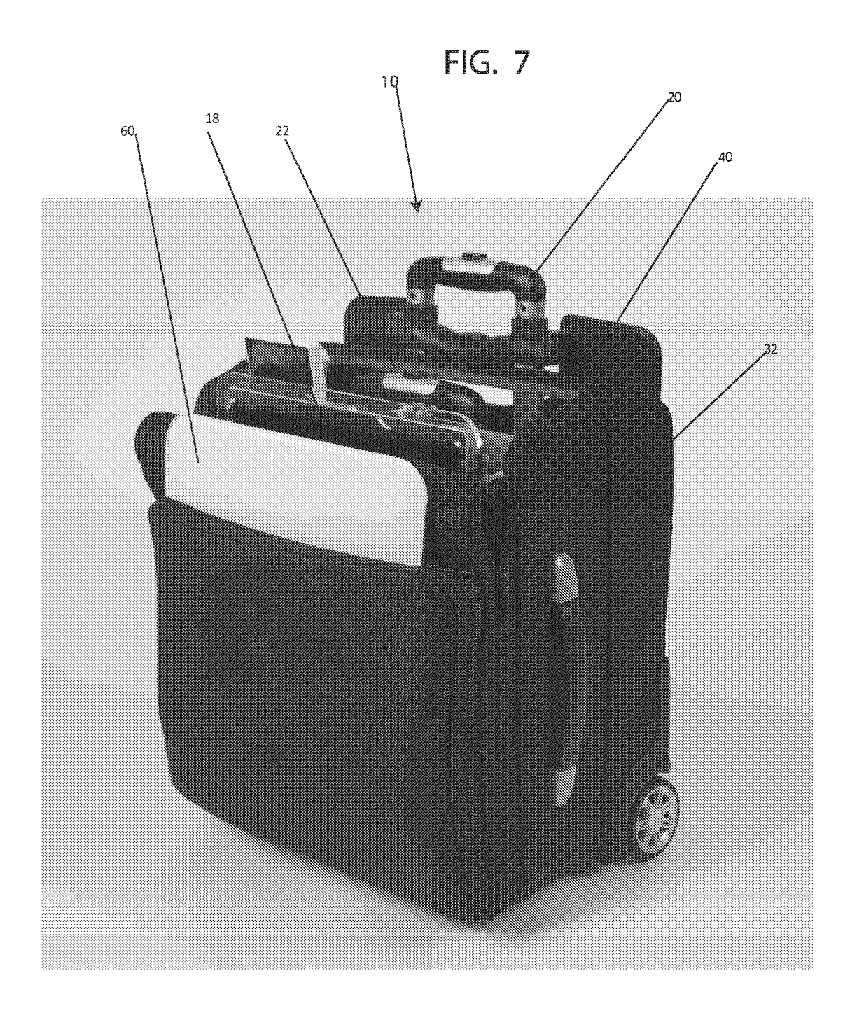

FIG. 7 shows the device 10 in a collapsed state. In this view, the device is shown collapsed into a bag with scale 60, screen 18 handles 22, and 20 as well as the other components collapsed into bag 32. Tray 40 is shown folded back into a back side of bag 32. In this collapsed form, the device is easily transportable and movable about an office or between offices as well. In at least one embodiment of the invention, the bag is small enough to be used as a standard carry on sized type luggage which allows this bag to be easily transported via air, rail, or auto.

FIG. 8 is a block diagram of the device which includes a main computer 80 having a microprocessor 81, a memory 83, and a transceiver 85 all coupled together on a motherboard such as motherboard 87. A plurality of input/output I/O connections (not shown) are available such as serial, USB, HDMI, VGA, are available for the connection of computer 80 to other peripheral devices. For example, coupled to this computer 80 are screen 16, screen 18, stethoscope 28, ear probe 93, thermometer 39.2, weight scale 60, speaker 26, camera/microphone 86, ultrasound 14, and blood pressure monitor 82.

Thus, this compact device is configured to be connected to or connectable to multiple different peripheral devices while also being highly transportable as well. This device then allows medical professionals to conduct a relatively thorough exam on an individual. The camera in the system allows for remote medical professionals to interview and see a patient from a remote location as well.

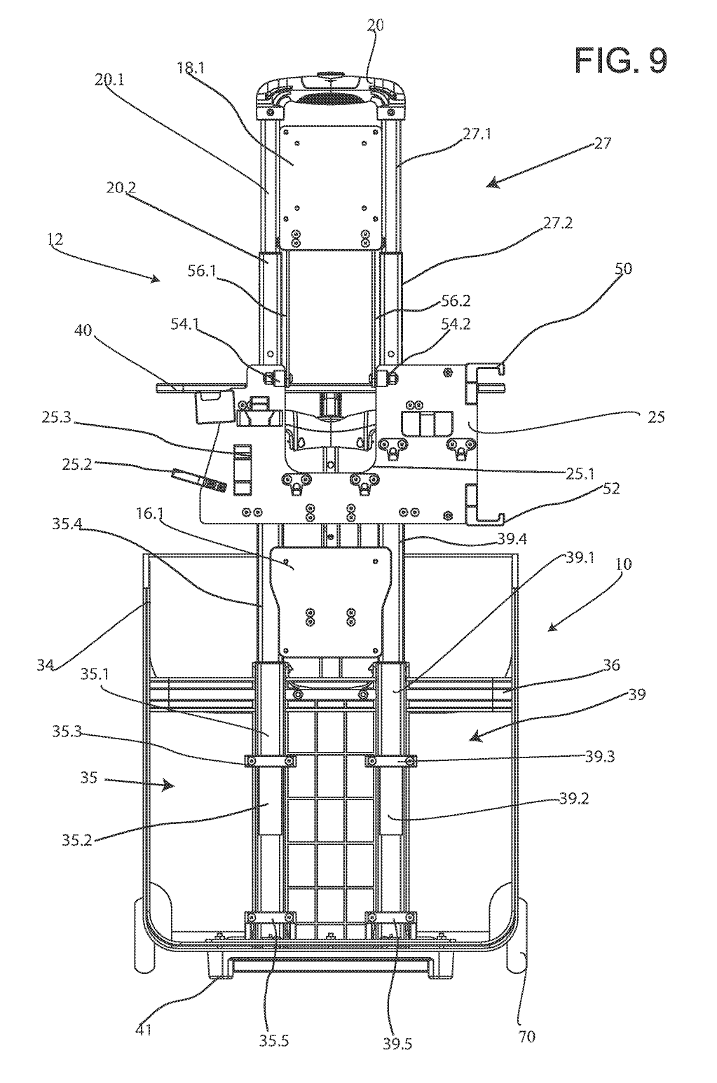

FIG. 9 is a front view of the device without screens. For example, there is shown the device 10 with handle 20 shown. There is also shown a back plate 18.1 which is configured to support screen 18 as well. Handle 20 has arms which are telescoping arms including a first section 20.1 a second section 20.2. In addition, an opposite arm 27.1 is telescoping into another section 27.2. Adjustable bolts 54.1 and 54.2 form a rotatable hinge for back plate 18.1. Back plate 18.1 is coupled to this hinge via arms 56.1 and 56.2.

Frame 25 includes a rounded U-shaped cut out 25.1 and also has brackets 25.2 and 25.3 coupled to it. These brackets are configured to hold peripheral devices. In addition, other brackets SO and 52 are coupled to frame 25 on an opposite side. Back tray 40 is shown extending out substantially horizontally from the rest of the device. Another back plate 16.1 is configured to support another screen such as screen 16.

A substantially rigid frame 34 is shown extending around a base section. This substantially rigid frame is substantially U-shaped and includes at least one reinforcing track 36.

A plurality of column like arms 35 and 39 including parts 35.1 and 39.1 as well as 35.2 and 39.2 are shown telescoping up into arms 35.4 and 39.4 These arms are held in place with support brackets 35.3 and 39.3 and 35.5 and 39.5 as well. At the bottom of the frame 34 is a housing 41 which is positioned between wheels 70.

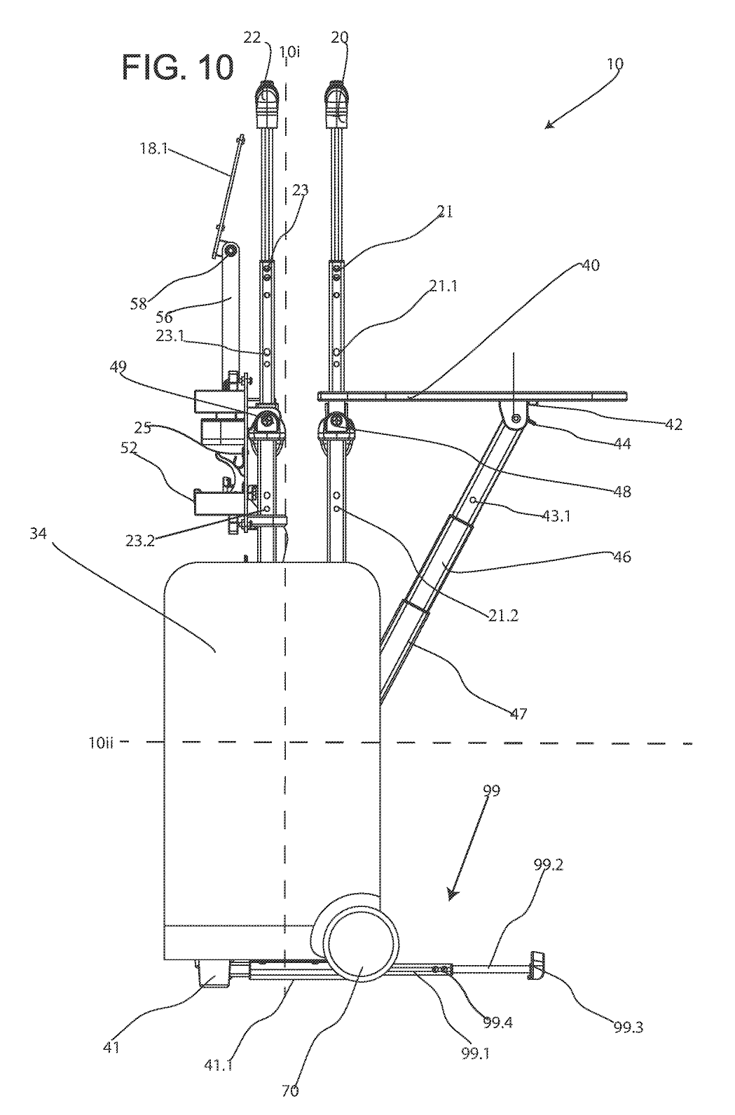

FIG. 10 is a side view of this embodiment. In this view, there is shown handles 20 and 22 along with line 10i which is a bisecting line. Line 10i is positioned between handles 20 and 22 so that these handles are positioned so that they are on either side of bisecting line 1 Oi. Bisecting line 1 Oi bisects the bag and the device. Back plate 18.1 is shown coupled to hinge 58 which is coupled to arm 56. In addition, arms 22 and 20 are shown as telescoping arms having locks 23 and 21 formed as spring loaded detents to allow these arms 22 and 20 to be selectively collapsed. For example, there is a first set of spring loaded detents forming locks 21 and 23, along with a second set of detents forming locks 23.1 and 21.1 as well. Furthermore, there is another set of locks 23.2 and 21.2 as well. Additional lower handle sections 48 and 49 are also shown as well.

Furthermore, a bracket 52 is shown extending out from frame 25 Frame 34 is shown coupled to arms 47, 46, and 43 wherein these arms are collapsible via locks such as locks 43.1. A button 42 is configured to collapse all of these arms 43,46, and 47 into each other with a single press of this button which acts on tab 44 to unlock the locks and collapse the arms. Under frame 34 is housing 41 which along with an arm housing 41.1 house arms 99.1, and 99.1 which are selectively locked in place via lock 99.4 along with top handle 99.3. These arms extend out along a substantially latitudinal axis 10ii which is transverse or substantially perpendicular to the longitudinal axis or bisecting axis 10i. Thus, handle 99 expands in a direction substantially perpendicular to the direction of expansion of handles 20 and 22.

The expansion of handle 99 allows for support of this device in a substantially horizontal manner when the device is expanded so that this can serve to prevent any rotational movement resulting from too much weight on tray 40 resulting in the device tipping over.

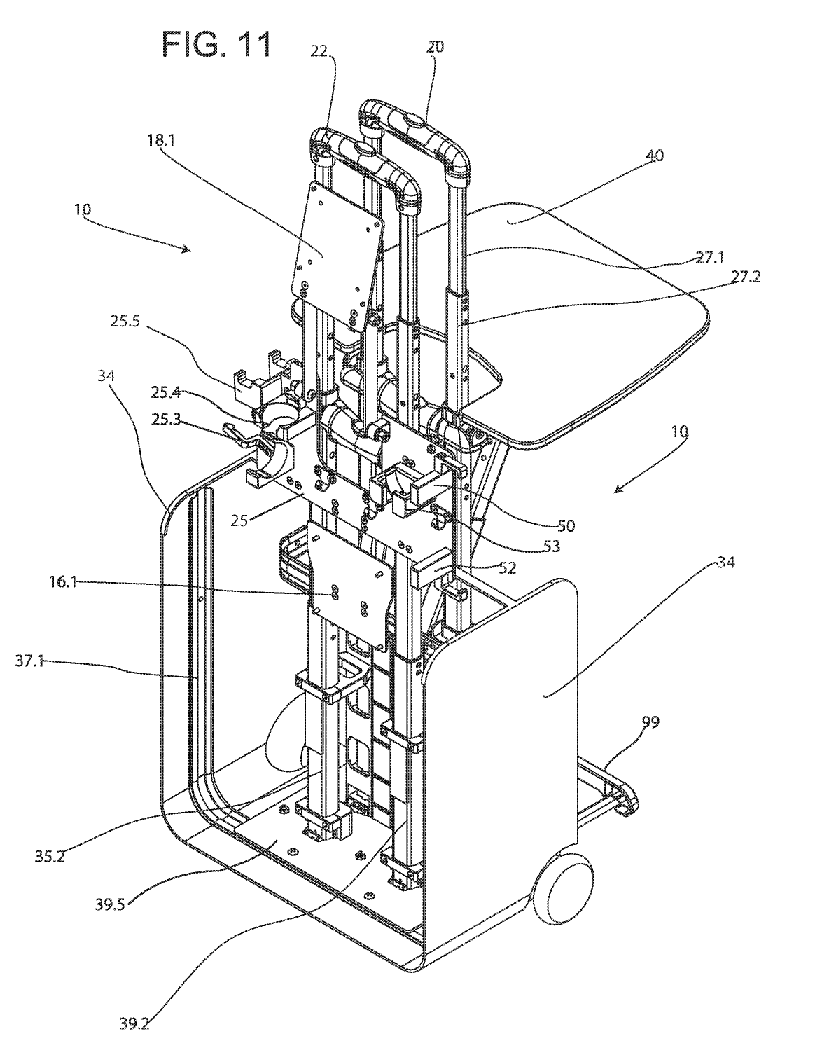

FIG. 11 also shows this handle 99 extending out from the back of the device as well. This view also shows handles 20 and 22, tray 40, plates 18.1 and 16.1 Arms 27.1 and 27.2 are shown as telescoping arms which collapse into each other and brackets 25.5 25.4, 25.3, as well as brackets SO, 52 and 53 are all coupled to frame 25. A track 37.1 is shown extending around the frame 34 in a U-shaped manner and coupled to base plate 39.5. Base plate 39.5 is coupled to arms 35.2 and 39.2 as well.

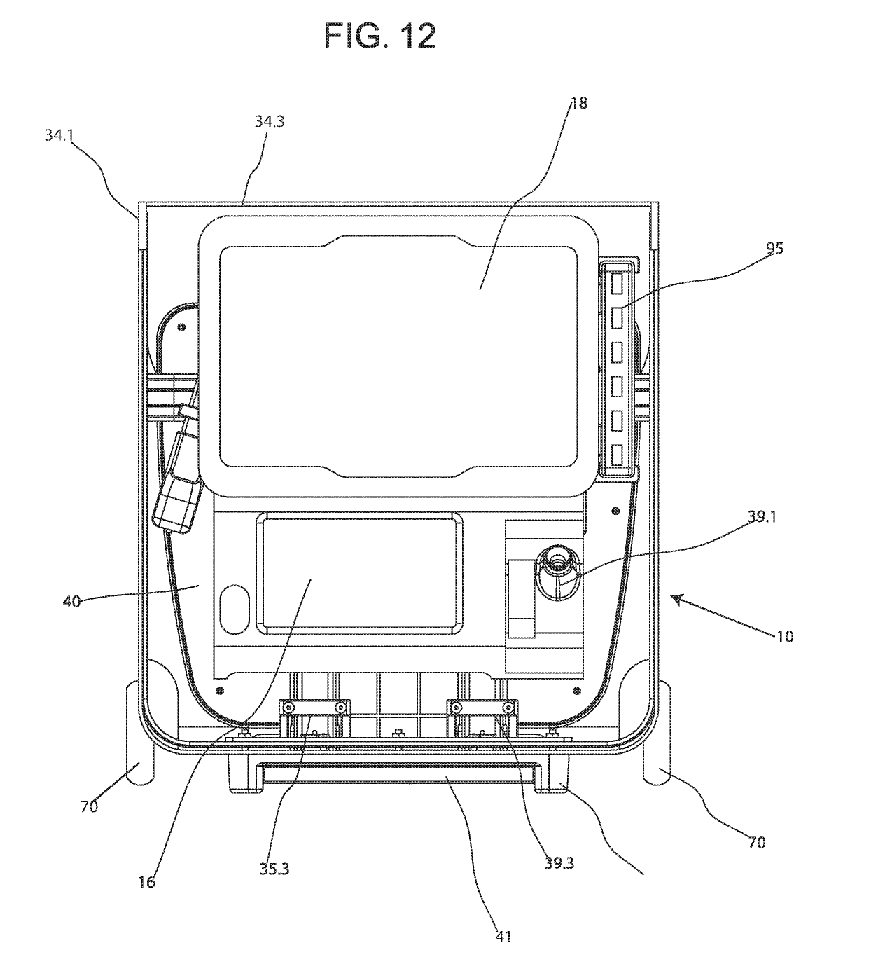

FIG. 12 shows a collapsed version of the device which shows screen 18 which is coupled to screen plate 18.1 (not shown). A top rim 34.1 is shown coupled to frame 34 along a top edge 34.3 A power strip 95 extends substantially vertically, substantially parallel to longitudinal axis 1 Oi. In this view tray 40 is shown along with main screen 16 which is coupled to screen plate 16.1. A housing 39.1 for a thermometer is shown positioned adjacent to screen 16. In addition, there are shown brackets 35.3 and 39.3 which support arms 35.2 and 39.2 as well. Below frame 34 is housing 41 which houses arm 99. Housing 41 is positioned between two wheels 70.

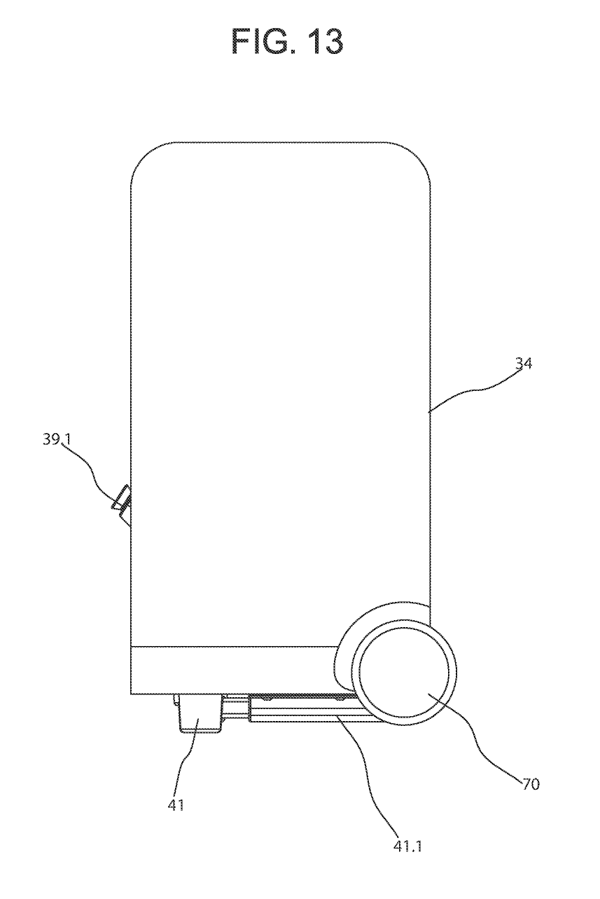

FIG. 13 is a side view of the device in a collapsed form showing housing 39.1 as well as frame 34. There is also shown wheels 70 as well as housing 41 positioned underneath frame 34. Additional housing 41.1 is shown housing the arms 99 as well.

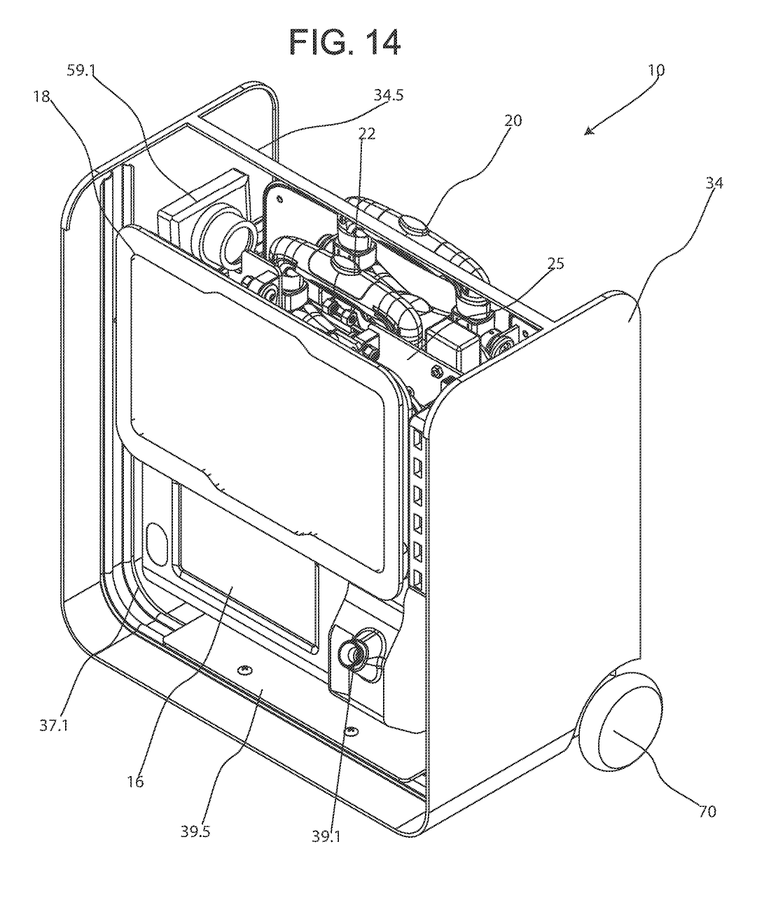

FIG. 14 shows a top, front, side view of the device 10 which shows frame 34 There is shown cross bar 34.5 which couples two sides of frame 34 together to provide stability. In addition, there is shown an additional bracket 59.1 which is coupled to frame 25 (See FIG. 9). This bracket can be used to house a stethoscope or other type of peripheral device. Track 37.1 is shown in frame 34 as well as well as bottom plate 39.5. Wheels 70 are also shown coupled to frame 34.

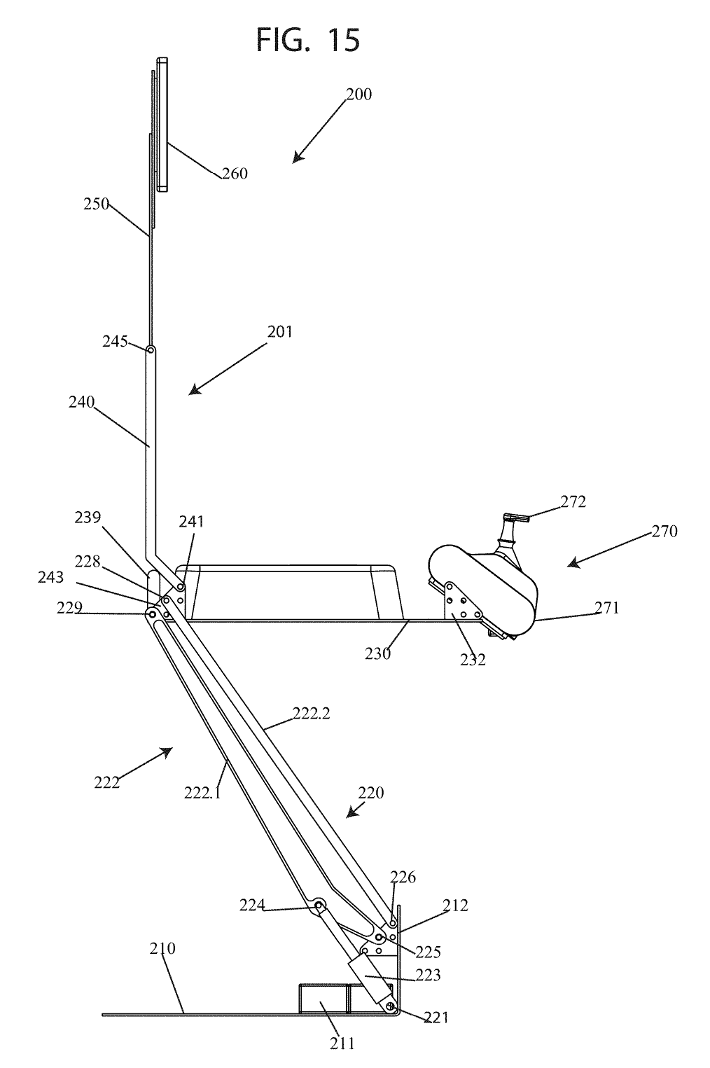

FIG. 15 shows another embodiment of the invention which relates to a portable mobile medical device or system 200. With this device there is a frame 201 which has a base 210 with a plurality of arms coupled to the base. In addition, there is a tray 230 and a screen 260 coupled to these arms. Inside of the tray or coupled to the tray 230 are a plurality of electronic and medical devices.

Coupled to base 210 is a power supply 211 which in one embodiment is a transformer configured to receive power from an outlet. In at least another embodiment, this power supply 211 is a battery pack and transformer combination. Coupled to base 210 is a first hinge 221. A spring such as a gas spring or piston 223 is coupled to hinge 221. Gas spring or piston 223 is configured to aid in the opening and closing of the device by biasing the station into an open position when a handle 239 is pulled when the device is in a closed position. At an opposite end to hinge 221 is another hinge 224. Arms 222 comprise arms 222.1 and 222.2. Arm 222.1 is coupled at a first end to hinge 224 and at a second end to hinge 229. Arm 222.1 is also coupled to a bracket 212 via hinge 225. Bracket 212 is a triangular shaped bracket that has at least two hinges 225 and 226 coupled to it. Thus arm 222.1 is coupled at one end via two different hinges 224 and 225. Arm 222.1 is thus angled so that the hinges 224 and 225 are offset from each other.

Arm 222.2 is coupled at one end to hinge 226 and at another end to hinge 228. Hinge 226 is coupled to bracket 212. Hinge 228 is also coupled to another substantially triangularly shaped bracket 243 as well. Bracket 243 also has hinge 241 coupled to it as well as tray 230 coupled to it as well. Tray 230 is essentially in the form of a table when extended out. While tray 230 is coupled at one end to bracket 243, it is coupled to another substantially triangularly shaped bracket 232 at another end. In addition, a handle 239 is coupled to bracket 243 as well.

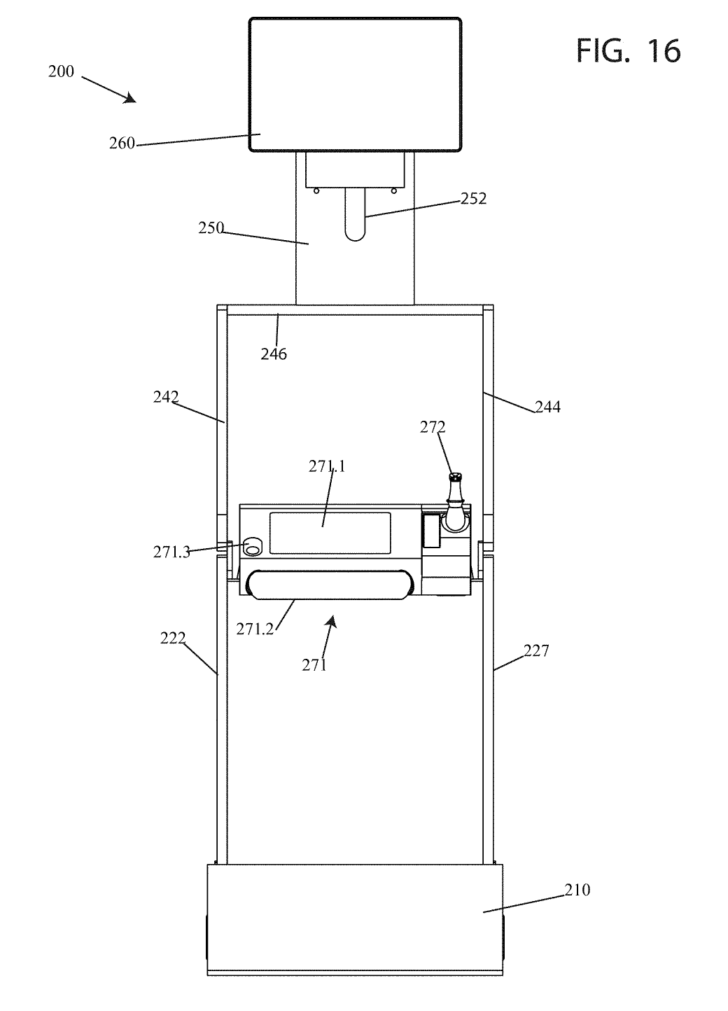

A set of arms 240 comprising upper arms when the device is in an extended position extend out from bracket 243. Arms 240 comprise a first set of arms 242 and a second set of arms 244 (See FIG. 16). Arms 240 are coupled at a first end to hinge 241 and at a second end to hinge 245. A bracket 250 is coupled at a first end to hinge 245 and at a second end to screen 260.

In addition, there are a plurality of medical devices 270 coupled to bracket 232. These medical devices include a at least a central computer 271, and a probe 272.

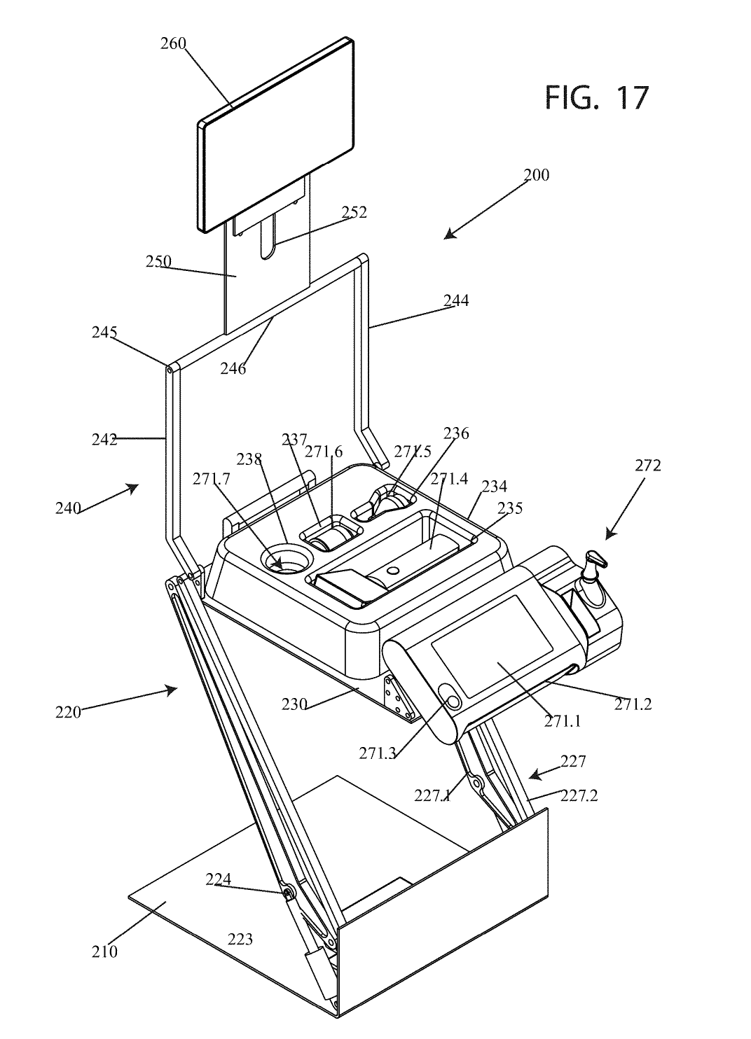

FIG. 16 is a front view of the device 200 which shows base 210 with arms 222 and 227 extending up therefrom. Arms 227 comprise arms 227.1 and 227.2 (See FIG. 17) wherein arms 227 are substantially identical to arms 222. There is shown central computer 271 having a pull out keyboard 271.2, and an on-off switch 271.3. Probe 272 is in communication with central computer 271 and is in communication with computer 271 in either a wired or wireless manner.

This view also shows arms 242 and 244 extending up to bracket 250. Bracket 250 has a slot 252 which allows screen 260 to be moved up or down.

FIG. 17 shows a top perspective view of the device 200 which shows both arms 222 and 227. This view also shows central computer 270 having a touch screen panel 271.1 and an on-off switch 271.3. Probe 272 is coupled to the central computer 270. In this view tray 234 is shown coupled to tray or table 230. Tray 234 has a plurality of containers such as a first container 235, a second container 236, a third container 237, and a fourth container 238. Inside of each of these containers are peripheral medical devices such as a first peripheral medical device 271.4, a second peripheral medical device 271.5 a third peripheral medical n device 271.6 and a fourth peripheral medical device 271.7. These different peripheral medical devices can include any one of a stethoscope, a thermometer, an ultrasound, a camera, a speaker and a blood pressure monitor.

A handle 239 is shown positioned adjacent to tray 234. As indicated above, when handle 239 is pulled up, it causes arms 222 and 224 to rotate clockwise about hinges 221, 225 and 226 while tray 230 rotates counter clockwise about hinge 229 away from arms 222 and 224. Meanwhile arms 240 including arms 242 and 244 rotate counter clockwise relative to hinge 241 thereby allowing for the opening of screen 260. Bracket 250 is rotatable about hinge 245 so that the screen can remain visible. Screen 260 can be in the form of a tablet such as a tablet computer. The tablet computer can comprise a microprocessor, a microphone, speakers, a camera and a screen.

In addition, coupled to arms 242 and 244 is an arm/cross beam 246 which is coupled to arms 242 and 244 via a hinge 245. Screen 260 is slidable in a slot 252 so that the entire device can be expanded up or easily collapsed down and then carried via handle 239. Base 210 then provides a cover for the collapsed device. This device can be fit inside of a bag and even coupled to a bag as well. The bag can be configured to have rolling wheels as well.

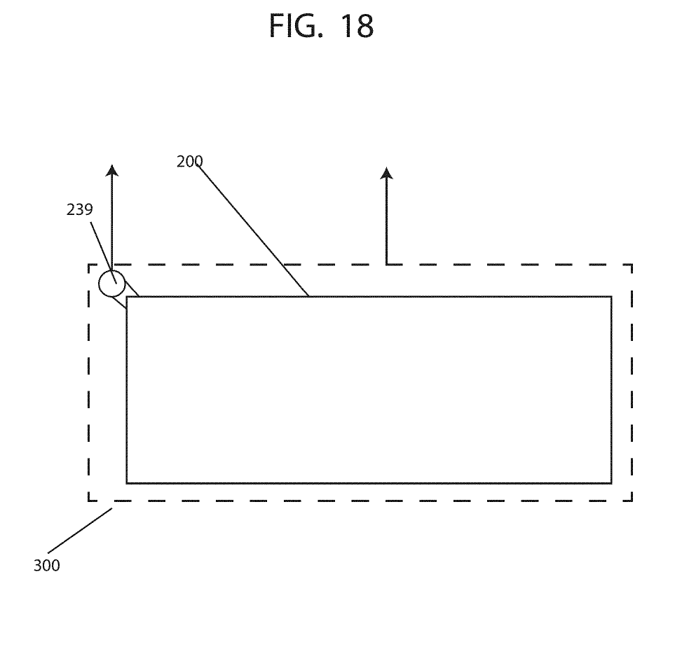

FIG. 18 shows the collapsed version of the device 200 disposed inside of a bag 300. The device is designed to either be transportable separate from a bag or collapsible inside of a bag. So that the device is easily openable and closable the device can rely on the spring such as gas spring 223 to either assist a user in opening up the device or closing the device in a more controlled manner. For example, when the user pulls on a handle in a direction shown by the arrow such as handle 239, the spring such as spring 223 is biased once it is moving to push open the arms 222 and 224 so as to raise up tray 230 and to allow for the extension of arms 240 so that the screen 260 and other components are is raised up and out of a bag such as bag 300.

In all, the different embodiments of the invention provide for a mobile, collapsible transportable device which can be used to provide extensive medical examinations to different personnel.

Accordingly, while at least one embodiment of the present invention have been shown and described, it is to be understood that many changes and modifications may be made thereunto without departing from the spirit and scope of the invention as defined in the appended claims.

What is claimed is:

1. A portable medical station comprising:
a main body;
a bag configured to selectively enclose at least a portion of the main body;
a tray coupled to the main body;
at least one handle coupled to the main body;
at least one screen coupled to the main body;
at least one spring coupled to the main body, wherein said spring is configured to aid in opening and closing said at least one computer into said bag;
at least one computer coupled to the main body, wherein the portable medical station is foldable inside of said bag so that the portable medical station is movable while being substantially folded inside of said bag.

2. The portable medical station as in claim 1, further comprising a tray hinge, wherein said tray is foldable out from said main body via said tray hinge.

3. The portable medical station as in claim 1, wherein said plurality of screens comprises a first screen which is configured as a video screen coupled to said at least one computer, and a second screen comprising a data readout screen coupled to said at least one computer.

4. The portable medical station as in claim 1, wherein said plurality of handles comprises a plurality of telescoping handles that are extendable out from said main body.

5. The portable medical station as in claim 1, wherein said at least one main body comprises a frame.

6. The portable medical station as in claim 1, further comprising at least one peripheral device.

7. The portable medical station as in claim 1, wherein said at least one peripheral device comprises at least one stethoscope.

8. The portable medical station as in claim 1, wherein said at least one peripheral device comprises at least one thermometer.

9. The portable medical station as in claim 1, wherein said at least one peripheral device comprises an ultrasound device.

10. The portable medical station as in claim 1, wherein said at least one peripheral device comprises at least one camera.

11. The portable medical station as in claim 1, wherein said at least one peripheral device comprises at least one speaker.

12. The portable medical station as in claim 1, wherein said at least one peripheral device comprises at least one blood pressure monitor.

13. The portable medical station as in claim 1, further comprising a plurality of arms extending out from said main body wherein a first end of said plurality of arms are coupled to said main body and a second end of said plurality of arms are coupled to said tray.

14. The portable medical station as in claim 1, wherein said at least one screen comprises a tablet computer.
